(19) United States
(12) Patent Application Publication
(10) Pub. No.: US 2018/0105595 A1
(43) Pub. Date: Apr. 19, 2018

(54) TIME AND SPACE ADJUSTABLE SYSTEM FOR INHIBITING PATHOLOGICAL TARGET CELLS

(71) Applicant: CARSGEN THERAPEUTICS LTD, Shanghai (CN)

(72) Inventors: Huamao WANG, Shanghai (CN); Bo SONG, Shanghai (CN); Peng WANG, Shanghai (CN)

(73) Assignee: CARSGEN THERAPEUTICS LTD, Shanghai (CN)

(21) Appl. No.: 15/322,033

(22) PCT Filed: Jun. 26, 2015
(86) PCT No.: PCT/CN2015/082460

§ 371 Date: Dec. 23, 2016

(30) Foreign Application Priority Data
Jun. 26, 2014 (CN) 201410299340.9

(51) Int. Cl. C07K 16/28; A61K 35/17; C07K 14/705; C07K 14/725; A61P 35/00

(52) U.S. Cl. CPC C07K 16/2863; A61K 35/17; C07K 14/70521; A61K 2039/505; C07K 14/7051; A61P 35/00; C07K 2317/622; C07K 14/70578

(57) ABSTRACT

The present invention provides a time and space adjustable system for inhibiting pathological target cells. The system comprises: (1) fusion protein, comprising polypeptide tags and binding molecules for specifically recognizing pathological target cells; and (2) chimeric antigen receptor immune effector cells, which express binding molecules for specifically recognizing the polypeptide tags. Disclosed is a technical solution based on a tumor-specific chimeric antigen receptor (CAR) technology, in which the immune effector cells can target pathological target cells only in the presence of a mediator, and the CAR immune effector cells can expand continuously and have killing effect on tumor cells; the CAR immune effector cells have no effect in the absence of a mediator.

### TECHNICAL FIELD

The present invention relates to the field of tumor immunology, and in particular, to a time and space-adjustable system for inhibiting pathological target cells.

### BACKGROUND

With the development of theory and technology of tumor immunology, the role of immune therapy in tumor treatment attracts more attention. T lymphocyte plays a main role in tumor immune response, and immune effector cells recently developed by employing gene modification technology for expressing tumor-specific chimeric antigen receptor (CAR) show targeting, killing activity, and durability, thereby providing a novel solution for adoptive cell immune therapy. For CAR, single chain antibody (scfv) or antibody fragment recognizing a tumor-related antigen (TAA) is subjected to gene recombination in vitro with activation sequence of T cells or NK cells, thereby forming recombinant plasmids. And T cells or NK cells purified and amplified are transfected in vitro through transfection technology, thereby obtaining CAR T cells or CAR NK cells. CAR mainly comprises antigen binding portion (extracellular domain) of TAA-specific antibody and T cell co-stimulatory structure (CD137 and CD28) and signaling structure (CD3ζ intracellular domain).

In studies, CAR T cells display excellent in vivo expansion, sustained activity, transformation into memory cells and anti-tumor effects. However, its toxic effects can not be ignored. In some tumors, CAR T cells recognize target antigens expressed by normal tissues or activate T cells to induce autoimmune responses. Persistent activated T cells and memory T cells can produce a substantial damage, for example toxicity to organ targets due to cross-reactivity.

Therefore, there is an urgent need in the art for methods, which can eliminate the toxicities of CAR T cells, but effectively and high-efficiently kill tumor cells.

### SUMMARY OF THE INVENTION

The object of the present invention is to provide a time and space-adjustable system for inhibiting pathological target cells.

In the first aspect of the present invention, a system for inhibiting pathological target cells is provided, comprising:

(1) a fusion protein, comprising a polypeptide tag and a binding molecule which specifically recognizes pathogenic target cells; and

(2) a chimeric antigen receptor (CAR) immune effector cell, expressing binding molecules which specifically recognize the polypeptide tag (including an antibody or ligand for the polypeptide tag).

In a preferred embodiment, the polypeptide tag is an unrelated antigen with low immunogenicity (including non-immunogenicity), which is of low or non-expression in non-tumor tissue.

In another preferred embodiment, the polypeptide tag is an endogenous or exogenous polypeptide.

In another preferred embodiment, the polypeptide tag is selected from (but not limited to), WTE, E-tag, Flag, Myc, His6, and the like.

In another preferred embodiment, the polypeptide tag is a WTE tag.

In another preferred embodiment, the polypeptide tag is a polypeptide encoded by the nucleotide sequence of SEQ ID NO: 38.

In another preferred embodiment, the polypeptide tag can be fused at the N-terminus or C-terminus of the binding molecule that specifically recognizes the pathological target cell, or fused at the N-terminus and C-terminus of the antibody.

In another preferred embodiment, the pathological target cell is a tumor cell, and the binding molecule that specifically recognizes the pathological target cell binds to tumor-associated antigen on the tumor cell.

In another preferred embodiment, the tumor-associated antigen is selected from (but not limited to):

EGFR, EGFRvIII, de4 EGFR, EpCAM, CD19, CD20, CD33, HER2, EphA2, IL13R, GD2, LMP1, Claudin 18.A2, PLAC1, NY-ESO-1, MAGE4, MUC1, MUC16, LeY, CEA, GPC3, Mesothelin, CAIX (Carbonic anhydrase IX), CD123, IL13R, EphA2.

In another preferred embodiment, the binding molecule is a ligand or antibody, and the antibody includes (but not limited to): Fab, F(ab′), F(ab′)₂, Fv, dAb, Fd, complementary determining region (CDR) fragment, single-chain antibody (scFv), bispecific single chain antibody, single chain phage antibody, bispecific double chain antibody, triple chain antibody, quadruplex chain antibody, monoclonal antibody.

In another preferred embodiment, the tumor includes (but not limited to): hepatocellular carcinoma, lung cancer, glioma, breast cancer, stomach cancer, prostate cancer, brain tumor, ovarian cancer, bone tumor, colon cancer, thyroid tumor, mediastinal tumor, intestine tumor, renal tumor, adrenal gland tumor, bladder tumor, malignant tumor Lymphoma, multiple myeloma, nervous system tumor, esophageal cancer, thymic mesothelioma, pancreatic cancer, leukemia, head and neck cancer, cervical cancer, skin cancer, melanoma, vaginal cancer, gallbladder cancer, malignant fibrous tissue tumor.

In another preferred embodiment, the immune effector cells include: T lymphocytes (including CD4⁺ or CD8⁺ T lymphocytes), NK cells.

In another preferred embodiment, the pathological target cells are tumor cells that express (preferably overexpress) EGFRvIII; and

the binding molecule that specifically recognizes pathogenic target cells is an antibody that specifically binds EGFRvIII, preferably a CH12 antibody.

In another preferred embodiment, the chimeric antigen receptor immune effector cell recombinantly expresses one or more selected from CD28 (preferably CD28a, CD28b), CD137, CD3ζ (preferably CD3ζ intracellular domain), CD27, CD8, CD19, CD134, CD20, FcRγ.

In another preferred embodiment, the chimeric antigen receptor immune effector cell comprises a construct comprising the following operably linked elements: encoding sequence for the binding sequence that specifically recognizes the polypeptide tag, CD8 hinge region, CD28a, CD28b, CD137, CD3ζ (preferably also comprising eGFP, F2A). Preferably, the elements in the construct are ligated in the following order (5′→3′): encoding sequence for the binding sequence that specifically recognizes the polypeptide tag, CD8 hinge region, CD28a, CD28b, CD137, CD3ζ (preferably also comprising (5′→3′) eGFP, F2A at 5′-end).

In another aspect of the present invention, use of any of the above mentioned systems is provided for preparing a medical cartridge for inhibiting pathological target cells. Preferably, the use is for non-therapeutic purpose.

In another aspect of the present invention, a kit is provided for preparing the medical cartridge is provided, wherein the kit includes:

(a) expression construct a comprising an expression cassette of a fusion protein (which can be expressed in immune cells), wherein the fusion protein comprises a polypeptide tag and a binding molecule that specifically recognizes pathological target cells;

(b) expression construct b comprising an expression cassette (which can be expressed in immune cells) which expresses a binding molecule that specifically recognizes the polypeptide tag (including an antibody or ligand that recognizes the polypeptide tag, etc.); and

(c) immune effector cells.

In a preferred embodiment, the pathological target cells are tumor cells, and the binding molecule that specifically recognizes pathological target cells binds to tumor-associated antigens on tumor cells; and/or the chimeric antigen receptor immune effector cell recombinantly expresses one or more selected from CD28 (preferably CD28a, CD28b), CD137, CD3ζ (preferably CD3ζ intracellular domain), CD27, CD8, CD19, CD134, CD20, FcRγ.

In another preferred embodiment, the expression construct a or expression construct b can be one or more vectors.

In another aspect of the present invention, a method for inhibiting pathological target cells is provided, including administering a subject the system for inhibiting pathological target cells.

In another aspect of the present invention, a method for time and space-adjustably inhibiting pathological target cells is provided, including: administering to a subject chimeric antigen receptor immune effector cells that express a binding molecule that specifically recognizes a polypeptide tag; and when it is necessary to inhibit pathological target cells, administering to a subject a fusion protein, wherein the fusion protein comprises a polypeptide tag and a binding molecule specifically recognizing the pathogenic target cells, thereby mediating immune effector cells for playing a role in killing pathological target cells.

In another aspect of the present invention, an isolated polypeptide (WTE tag) is provided, wherein the amino acid sequence of the polypeptide is encoded by the nucleotide sequence of SEQ ID NO: 38.

In another aspect of the present invention, an isolated polynucleotide is provided, wherein the nucleotide sequence of the polynucleotide is shown in SEQ ID NO: 38 or a degenerate sequence thereof.

In another aspect of the present invention, a single chain antibody that specifically binds to the polypeptide (WTE tag) is provided, wherein the single chain antibody is encoded by the nucleotide sequence of SEQ ID NO: 35.

In another aspect of the present invention, a polynucleotide encoding the single chain antibody is provided, wherein the nucleotide sequence of the polynucleotide is shown in SEQ ID NO: 35 or a degenerate sequence thereof.

Other aspects of the present invention will become apparent to those skilled in the art from the disclosure herein.

### BRIEF DESCRIPTION OF THE DRAWINGS

FIG. 1 shows a schematic figure of the structure of pK/WTE-CH12L expression vector.

FIG. 2 shows a schematic figure of the structure of pK/WTE-CH12H expression vector.

FIG. 3 shows the structure of antibody WTE-CH12.

FIG. 4 shows a schematic figure of the structure of lentiviral vector pWPT/eGFP-2D8 (anti-WTE)-CD28a-CD28b-CD137-CD3ζ comprising CAR encoding sequence.

FIG. 5 shows detection through sodium dodecyl sulfate-polyacrylamide gel electrophoresis (SDS-PAGE) of purified WTE-CH12 antibody, wherein M is the molecular weight marker and Lane 1 represents the purified WTE-CH12 antibody.

FIG. 6A shows determination of specific binding of WTE-CH12 antibody to U87MG tumor cells by fluorescence activated cell sorter (FACS).

FIG. 6B shows determination of specific binding of WTE-CH12 antibody to U87MG-EGFRvIII tumor cells by fluorescence activated cell sorter (FACS).

FIG. 6C shows determination of specific binding of WTE-CH12 antibody to Huh-7 tumor cells by fluorescence activated cell sorter (FACS).

FIG. 6D shows determination of specific binding of WTE-CH12 antibody to Huh-7-EGFRvIII tumor cells by fluorescence activated cell sorter (FACS).

FIG. 7A shows the proportion of CAR⁺ T cells, after infection of lentiviral vector, by fluorescence activated cell sorter (FACS).

FIG. 7B shows the proportion of CAR⁺CD4⁺ T cells, after infection of lentiviral vector, by fluorescence activated cell sorter (FACS).

FIG. 7C shows the proportion of CAR⁺CD8⁺ T cells by fluorescence activated cell sorter (FACS).

FIG. 8A shows comparison of the cytotoxicity of CAR⁺ T cells induced by WTE-CH12 antibody serially diluted in gradient on U87MG, U87MG-EGFRvIII tumor cells.

FIG. 8B shows comparison of the cytotoxicity of CAR⁺ T cells induced by WTE-CH12 antibody serially diluted in gradient on Huh-7, Huh-7-EGFRvIII tumor cells.

FIG. 9 shows an in vitro competitive inhibition assay of WTE polypeptide on toxicity of T lymphocytes expressing chimeric antigen receptor on tumor cells mediated by WTE-CH12.

FIG. 10 shows an assay of anti-tumor activity of treatment group of WTE-CH12 antibody-induced CAR⁺ T cells and control group through NOD/SCID tumor (U87MG-EGFRvIII) mouse mode.

FIG. 11 shows an assay of anti-tumor activity of treatment group of WTE-CH12 antibody-induced CAR⁺ T cells and control group through NOD/SCID tumor (Huh-7-EGFRvIII) mouse mode.

### EMBODIMENTS FOR CARRYING OUT THE INVENTION

Upon extensive study, the present inventors have disclosed a method for space and time-adjustably inhibiting pathological target cells based on a tumor-specific chimeric antigen receptor (CAR) technology. CAR immune effector cells (such as CAR T cells) engineered by the present inventors can target pathological target cells only in the presence of a mediator, thereby achieving the continuous amplification of CAR immune effector cells and exerting the killing effect on tumor cells; while in the absence of mediated mediators, the CAR immune effector cells do not function (or merely less function). The present invention provides a solution for avoiding toxic effects from the in vivo continuous amplification of CAR immune effecting cells and cross reaction with the normal tissues.

In the present invention, the binding molecule recognizing a pathogenic target cell associated antigen (e.g., tumor associated antigen) in a chimeric antigen receptor (CAR) immune effector cell is replaced with a binding molecule (e.g., single chain antibody) that recognizes a polypeptide tag (unrelated antigen), and the binding molecule that recognizes pathogenic target cell-associated antigens is fused with the polypeptide tag, so as to obtain a fusion protein. The mode of separating the binding molecules that recognize pathogenic target cell-associated antigens from conventional CAR immune effector cells can be used to selectively regulate the recognition signal. After pathological target cells are removed, withdrawal of the fusion protein results in the loss of targeting activity for the CAR immune effector cells in a patient, and the blocking of this signal prevented the CAR T cells from recognizing the low-expression target antigen of the normal tissues and continuous expansion, so as to solve the toxic effects which may be caused by such problem.

The term “chimeric antigen receptor (CAR) immune effector cell” is well known in the art, and means an immune effector cell which expresses a tumor-specific chimeric antigen receptor using genetic modification techniques, exhibits some targeting, killing activity and persistence in vitro and in clinical trials, and is an adoptive cellular immunotherapy. The immune effector cells include, for example, T cells and NK cells.

Conventional methods for preparing “chimeric antigen receptor immune effector cells” are known to those skilled in the art and include expressing intracellular domains of intracellular co-stimulatory cell molecules, such as one or more of CD28 (preferably CD28a, CD28b), CD137, CD27, CD3ζ (preferably CD3ζ intracellular domain), CD8, CD19, CD134, CD20, FcRγ. Upon binding to a corresponding ligand, the second signal of immune effector cells can be activated, the proliferation ability of immune cells and the secretion of cytokines can be enhanced, and the survival time of activated immune cells can be prolonged.

In the present invention, the pathological target cells may be various harmful cells in the body, which are harmful to health and needed to be removed from the body. The pathological target cells include tumor cells. Any tumor known in the art may be included in the present invention so long as the tumor is capable of expressing tumor-associated antigens lowly expressed in normal tissues.

For example, the tumor includes, but not limited to, liver cancer, lung cancer, glioma, breast cancer, stomach cancer, prostate cancer, brain tumor, ovarian cancer, bone tumor, colon cancer, thyroid tumor, mediastinal tumor, renal cancer, adrenal tumor, bladder tumor, testicular tumor, malignant lymphoma, multiple myeloma, nervous system tumor, esophageal cancer, thymic mesothelioma, pancreatic cancer, leukemia, head and neck cancer, cervical cancer, skin cancer, melanoma, vaginal epithelial cancer, gallbladder cancer, malignant fibrous histiocytoma.

For example, the tumor-associated antigen includes (but not limited to): EGFR, EGFRvIII, de4 EGFR, EpCAM, CD19, CD20, CD33, HER2, EphA2, IL13R, GD2, LMP1, Claudin 18.A2, PLAC1, NY-ESO-1, MAGE4, MUC1, MUC16, LeY, CEA, GPC3, Mesothelin, CAIX (Carbonic anhydrase IX), CD123, IL13R, EphA2.

In the present invention, the polypeptide tag is an antigen which is in low expression (negligible expression) or non-expression in a non-pathological tissue, has a low immunogenicity, does not induce significant immunity in vivo, and may be an endogenous or exogenous polypeptide. Any unrelated antigen that meets the above requirements may be included in the present invention, such as, but not limited to: WTE, E-tag, Flag, Myc, His6 and the like.

The “binding molecule that recognizes a polypeptide tag” is a binding molecule that specifically recognizes or binds to the polypeptide tag, and can be a ligand or antibody. The antibody includes (but not limited to): Fab, F(ab′), F(ab′)₂, Fv, dAb, Fd, complementary determining region (CDR) fragment, single-chain antibody (scFv), bispecific single chain antibody, single chain phage antibody, bispecific double chain antibody, triple chain antibody, quadruplex chain antibody, monoclonal antibody. Preferably, the antibody is single-chain antibody.

The binding molecule that specifically recognizes the pathological target cells exerts the action of targeting the pathological target cells. After the polypeptide tag binds to the binding molecule which recognizes the polypeptide label, the binding molecule which specifically recognizes the pathological carries the immune effector cell to the pathological target cells when targets the pathological target cell, thereby exerting killing effects.

The “binding molecule that specifically recognizes pathological target cells” can be any binding molecule that specifically recognizes pathogenic target cell associated antigens. Clinically, it is possible to determine which binding molecule is to be used according to the type of pathological target cells to be killed. For example, when the pathological target cell is a glioma cell (e.g. U87MG) or hepatoma cell (Huh-7) which specifically expresses EGFRvIII, application of an antibody which specifically binds to EGFRvIII is suitable.

In particular embodiments of the present invention, the single chain antibody recognizing tumor antigen, EGFRvIII in a CAR T cell is replaced with a single chain antibody that recognizes a unrelated antigen (polypeptide WTE), and monoclonal antibody CH12 recognizing tumor associated antigen (EGFRvIII) is linked to the unrelated antigen polypeptide WTE (derived from amino acids 1189-1210 of EGFR NM_005228 intracellular segment) via a linking peptide (see SEQ ID NO: 44, SEQ ID NO: 47 in U.S. Pat. No. 7,612,181), for expressing and preparing the recombinant protein. The mode of separating the antibody that recognize target antigens from conventional CAR T can be used to selectively regulate the recognition signal. After tumors are removed, withdrawal of anti-EGFRvIII antibody WTE-CH12 results in the loss of targeting activity for the CAR T cells in a patient, and the blocking of this signal prevented the CAR T cells from recognizing the low-expression target antigen of the normal tissues and continuous expansion.

The present invention also relates to a medical cartridge comprising a system for inhibiting pathological target cells, wherein the medical cartridge comprises (1) a fusion protein comprising a polypeptide tag and a binding molecule that specifically recognizes pathogenic target cells; and (2) a chimeric antigen receptor immune effector cell which expresses a binding molecule that specifically recognizes the polypeptide tag. The medical cartridge may also contain instructions for using the medical cartridge.

The invention will be further illustrated with reference to the following specific examples. It is to be understood that these examples are only intended to illustrate the invention, but not to limit the scope of the invention. For the experimental methods in the following examples without particular conditions, they are performed under routine conditions, such as conditions described in Sambrook et al., Molecular Cloning: A Laboratory Manual, New York: Cold Spring Harbor Laboratory Press, 1989, or as instructed by the manufacturer.

### Example 1. Construction of Recombinant Plasmid for Anti-Human EGFRVIII WTE-CH12 Antibody of the Present Invention

1. Amplification of Nucleic Acid Fragments

(1) Antibody pH/CH12 was used as a template (SEQ ID NO: 36). CH12VH fragment was amplified through PCR by using upstream primer 5′-gatgtgcagcttcaggagtcggg-3′ (SEQ ID NO: 1) and downstream primer 5′-acaataatatgtggctgtg tcc-3′ (SEQ ID NO: 2). PCR amplification conditions were: pre-denaturation at 94° C. for 4 min; denaturation at 94° C. for 40 s; annealing at 58° C. for 40 s; extension at 68° C. for 40 s; 27 cycles; followed by a total extension at 68° C. for 10 min. The size of the amplified product was 288 bp, which was in agreement with the expected size.

(2) Amplification of heavy chain signal peptide-WTE fragment with primers as follows:

(SEQ ID NO: 3)
5′-cctagctagccaccatgagagtgctgattcttttgtggctgttca
cagcctttcct-3′,
(SEQ ID NO: 4)
5′-agctgtggagccagacaggaaaccaggaaaggctgtgaacagcca
c-3′,
(SEQ ID NO: 5)
5′-ggtttcctgtctggctccacagctgaaaatgcagaatacctaagg
gtcgcg-3′,
(SEQ ID NO: 6)
5′-tgctccaataaattcactgctttgtggcgcgacccttaggtattc
tgcattttc-3′,
(SEQ ID NO: 7)
5′-ccacaaagcagtgaatttattggagcagcatcaaccaaaggtcct
gatgtg-3′,
(SEQ ID NO: 8)
5′-ctcctgaagctgcacatcaggacctttggttgatgc-3′;

In the first step, Overlap PCR was used, wherein SEQ ID NO: 3-SEQ ID NO: 8 were used as primers for synthesizing WTE fragment (SEQ ID NO:38) and heavy chain signal peptide sequence (SEQ ID NO:39). PCR amplification conditions were: pre-denaturation at 94° C. for 4 min; denaturation at 94° C. for 40 s; annealing at 58° C. for 40 s; extension at 68° C. for 40 s; 7 cycles; followed by a total extension at 68° C. for 10 min.

In the second step, PCR was performed, wherein products from Overlap PCR in the first step were used as templates, and SEQ ID NO: 3 and SEQ ID NO: 8 were used as upstream and downstream primers respectively for amplifying heavy chain signal peptide-WTE fragment. PCR amplification conditions were: pre-denaturation at 94° C. for 4 min; denaturation at 94° C. for 40 s; annealing at 58° C. for 40 s; extension at 68° C. for 40 s; 27 cycles; followed by a total extension at 68° C. for 10 min. The size of the amplified product was 134 bp, which was in agreement with the expected size.

(3) Antibody pK/CH12 was used as a template (SEQ ID NO: 37). CH12Vk fragment was amplified through PCR by using upstream primer 5′-gacatcctgatgacccaatctcc-3′ (SEQ ID NO: 9) and downstream primer 5′-gaagacagatggtgcagccac-3′ (SEQ ID NO: 10). PCR amplification conditions were: pre-denaturation at 94° C. for 4 min; denaturation at 94° C. for 40 s; annealing at 55° C. for 40 s; extension at 68° C. for 40 s; 27 cycles; followed by a total extension at 68° C. for 10 min. The size of the amplified product was 348 bp, which was in agreement with the expected size.

(4) Amplification of light chain signal peptide-WTE fragment with primers as follows:

(SEQ ID NO: 11)
5′-gatcgatatccaccatggacatgatggtccttgctcagtttcttgca
ttcttgttg-3′;
(SEQ ID NO: 12)
5′-aaaccaaagcaacaagaatgcaagaaactgagcaaggaccatcatgt
cc-3′;
(SEQ ID NO: 13)
5′-ctttggtttccaggtgcaagatgtggctccacagctgaaaatgcaga
atacc-3′;
(SEQ ID NO: 14)
5′-tggcgcgacccttaggtattctgcattttcagctgtggagccacatc
ttgcacctgg-3′;
(SEQ ID NO: 15)
5′-taagggtcgcgccacaaagcagtgaatttattggagcaacggtggct
gcaccagac-3′;
(SEQ ID NO: 16)
5′-ttgggtcatcaggatgtctggtgcagccaccgttgctccaataaatt
cactgctttg-3′;

Overlap PCR was employed, wherein SEQ ID NO: 11-SEQ ID NO: 16 were used as primers for synthesizing WTE fragment and light chain signal peptide sequence (SEQ ID NO: 40). In the first step, Overlap PCR amplification conditions were: pre-denaturation at 94° C. for 4 min; denaturation at 94° C. for 40 s; annealing at 58° C. for 40 s; extension at 68° C. for 40 s; 7 cycles; followed by a total extension at 68° C. for 10 min.

In the second step, PCR was performed, wherein products from Overlap PCR in the first step were used as templates, and SEQ ID NO: 11 and SEQ ID NO: 16 were used as upstream and downstream primers respectively for amplifying light chain signal peptide-WTE fragment. PCR amplification conditions were: pre-denaturation at 94° C. for 4 min; denaturation at 94° C. for 40 s; annealing at 58° C. for 40 s; extension at 68° C. for 40 s; 27 cycles; followed by a total extension at 68° C. for 10 min. The size of the amplified product was 179 bp, which was in agreement with the expected size.

2. Splicing of Nucleic Acid Fragment

(1) For splicing of heavy chain signal peptide-WTE-CH12VH fragment, upstream primer (SEQ ID NO: 3) and downstream primer 5′-acaataatatgtggctgtgtcc-3′ (SEQ ID NO: 2) were used in splicing to obtain heavy chain signal peptide-WTE-CH12VH; and splicing conditions were: pre-denaturation of heavy chain signal peptide-WTE (50 ng)+CH12VH (50 ng) at 94° C. for 4 min; denaturation at 94° C. for 30 s; annealing at 60° C. for 30 s; extension at 68° C. for 30 s; 7 cycles; followed by a total extension at 68° C. for 10 min; DNA polymerase and upstream and downstream primers were supplemented, and afterwards PCR amplification was performed for 25 cycles; and PCR amplification conditions were: pre-denaturation at 94° C. for 4 min; denaturation at 94° C. for 30 s; annealing at 60° C. for 30 s; extension at 68° C. for 30 s; 25 cycles; followed by a total extension at 68° C. for 10 min. The amplified product was confirmed by agarose gel electrophoresis to comply with the theoretical size, 441 bp.

(2) Splicing conditions for light chain signal peptide-WTE-CH12Vk fragment were: pre-denaturation of light chain signal peptide-WTE (50 ng)+CH12Vk (50 ng) at 94° C. for 4 min; denaturation at 94° C. for 30 s; annealing at 60° C. for 30 s; extension at 68° C. for 30 s; 7 cycles; followed by a total extension at 68° C. for 10 min; DNA polymerase and upstream primer 5′-gatcgatatccaccatggacatgatggtccttgctcagtttcttgcattcttgttg-3′ (SEQ ID NO:11) and downstream primer 5′-gaagacagatggtgcagccac-3′ (SEQ ID NO:10) were supplemented, and afterwards PCR amplification was performed for 25 cycles; thereby obtaining light chain signal peptide-WTE-CH12Vk. And amplification conditions were: pre-denaturation at 94° C. for 4 min; denaturation at 94° C. for 30 s; annealing at 60° C. for 30 s; extension at 68° C. for 30 s; 25 cycles; followed by a total extension at 68° C. for 10 min. The amplified product was confirmed by agarose gel electrophoresis to comply with the theoretical size, 509 bp.

3. Construction of Expression Vector Comprising Nucleotide Sequence Encoding WTE-CH12 Antibody

(1) Construction of pH/WTE-CH12H Vector

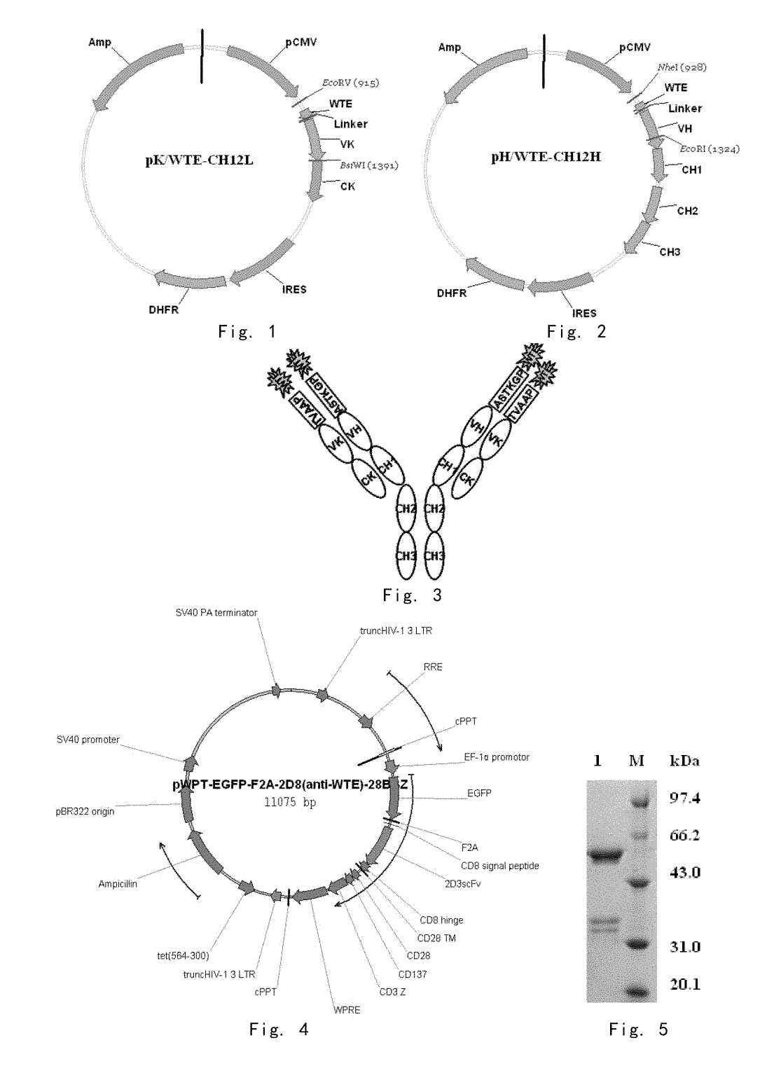

The sequences heavy-chain signal peptide-WTE-CH12VH and pH/CH12 obtained by amplification were digested with restriction endonucleases NheI/EcoRI, and double-digestion was performed according to the reaction conditions suggested by the supplier (New England Biolabs, NEB). Upon double digestion, heavy chain signal peptide-WTE-CH12 VH fragment and pH/CH12 vector fragment were then ligated with T4 DNA ligase according to the reaction conditions suggested by the supplier (NEB), thereby cloning the nucleotide sequence encoding WTE-CH12 VH antibody polypeptide into the vector. The resulting new vector containing the coding sequence of WTE-CH12 VH antibody polypeptide was named as pH/WTE-CH12H and its structure is shown in FIG. 2.

(2) Construction of pK/WTE-CH12L Vector

The sequences light-chain signal peptide-WTE-CH12Vk and pK/CH12 obtained by amplification were digested with restriction endonucleases EcoRV/BsiWI, and double-digestion was performed according to the reaction conditions suggested by the supplier (New England Biolabs, NEB). Upon double digestion, heavy chain signal peptide-WTE-CH12 VK fragment and pK/CH12 vector fragment were then ligated with T4 DNA ligase according to the reaction conditions suggested by the supplier (NEB), thereby cloning the nucleotide sequence encoding WTE-CH12 VK antibody polypeptide into the vector. The resulting new vector containing the coding sequence of WTE-CH12 VK antibody polypeptide was named as pK/WTE-CH12L and its structure is shown in FIG. 1.

### Example 2. Expression and Purification of Anti-Human EGFRvIII WTE-CH12 Antibody

1. Expression of Anti-Human EGFRvIII WTE-CH12 Antibody

Free-Style 293-F cells (purchased from Invitrogen) were used on the expression of antibody, suspension culture and transfection were performed according to the specification of FreeStyle™ 293 Expression System. Specifically, the cell density was adjusted to 1×10⁶ cells/mL before transfection, the cells were allowed to disperse without agglomeration, and cell viability was determined to be >95% by trypan blue staining. Transfection procedure: 52 μg of recombinant plasmid, pH/WTE-CH12H and 48 μg of pK/WTE-CH12K (molar ratio 1:1) and 200 μL of Free-Style 293-F cell liposome transfection reagent “293fectin” were diluted with Opti-MEM to 3.33 mL. After standing for 5 mins, the plasmids were slowly mixed with the transfection reagent, and incubated at room temperature for 20 min at room temperature to form a DNA-fectin mixture. Then, 93.3 mL of Free-Style 293-F cells (density: 1×10⁶ cells/mL) were added to the mixture to the final volume of 100 mL, and cultured at 37° C., 8% CO₂ and 130 r/min in a shake flask. After 7 days, the supernatant was obtained by centrifugation for purification of antibody in the next step.

2. Purification of Anti-Human EGFRvIII WTE-CH12 Antibody

Protein G affinity chromatography column (Protein G Sepharose Fast Flow from GE Healthcare) was used in the purification of antibody. Specifically, Protein G affinity column was warmed to room temperature, and the column was equilibrated by 5 column volumes of PBS. The supernatant obtained in step 1 was loaded onto the column at the flow rate of 3 ml/min. Upon loading, the column was equilibrated by 5 column volumes of PBS. The column was eluted with pH 2.7, 0.1 M glycine hydrochloride solution, and the eluate was neutralized by addition of 1/10 volume of 1 M NaH₂PO₄ solution, pH 9.0. The purified sample was desalted on a desalting column (Sephadex G-25F from GE). The desalted sample was filtered through a 0.22 um filter and stored, thereby obtaining the solution of purified antibody. The structure of the obtained antibody is shown in FIG. 3, and the purification results are shown in FIG. 5. The antibody with a purity of more than 95% is obtained by one-step purification method, which is abbreviated as WTE-CH12 antibody.

### Example 3. Detection of Binding Activity of Anti-Human EGFRvIII WTE-CH12 Antibody to Tumor Cells

The binding capacity of WTE-CH12 antibody to EGFRVIII was analyzed by a fluorescence activated cell sorter (FACS, commonly referred to as flow cytometry) (FACScalibur, BD).

Specifically, U87MG (purchased from ATCC), U87MG-EGFRvIII (U87-EGFRvIII cells in WO/2011/035465), Huh-7 (purchased from ATCC), Huh-7-EGFRvIII (method for transferring an EGFRvIII-encoding gene into Huh-7 cell can be found in Huamao Wang, et al., Epidermal growth factor receptor vIII enhances tumorigenicity and resistance to 5-fluorouracil in human hepatocellular carcinoma. Cancer Letters 279 (2009) 30-38.) at logarithmic growth phase were taken, inoculated into a 6 cm Petri dish and incubated at 37° C. in an incubator overnight. The cells were digested with 10 mM EDTA, and the cells were collected by centrifugation at 200 g×5 min. The cells were resuspended in 1% phosphate buffer containing calf bovine serum (NBS PBS) at a concentration of 5×10⁶/mL and added into a FACS tube at 100 μl/tube. The cells were centrifuged at 200 g×5 min, and the supernatant was discarded. Blank control PBS and antibody WTE-CH12 to be tested were added to two tubes (100 μl per tube) respectively, and the final concentration of each antibody was 5 μg/ml. The tubes were placed into an ice bath, and after 45 minutes, 2 ml 1% NBS PBS was added into each tube, centrifuged at 200 g×5 min, for two times in total. The supernatant was discarded and 1:50 dilution of goat anti-human-FITC antibody (purchased from Shanghai Yeli Biotech Co., Ltd.) was added, 100 μl per tube. After placing into an ice bath for 45 minutes, 2 ml of 1% NBS PBS was added to each tube and centrifuged at 200 g×5 min for two times. The supernatant was discarded and resuspended in 300 μl of 1% NBS PBS and detected by flow cytometry. Data were analyzed using WinMDI 2.9, a flow cytometric data analysis software.

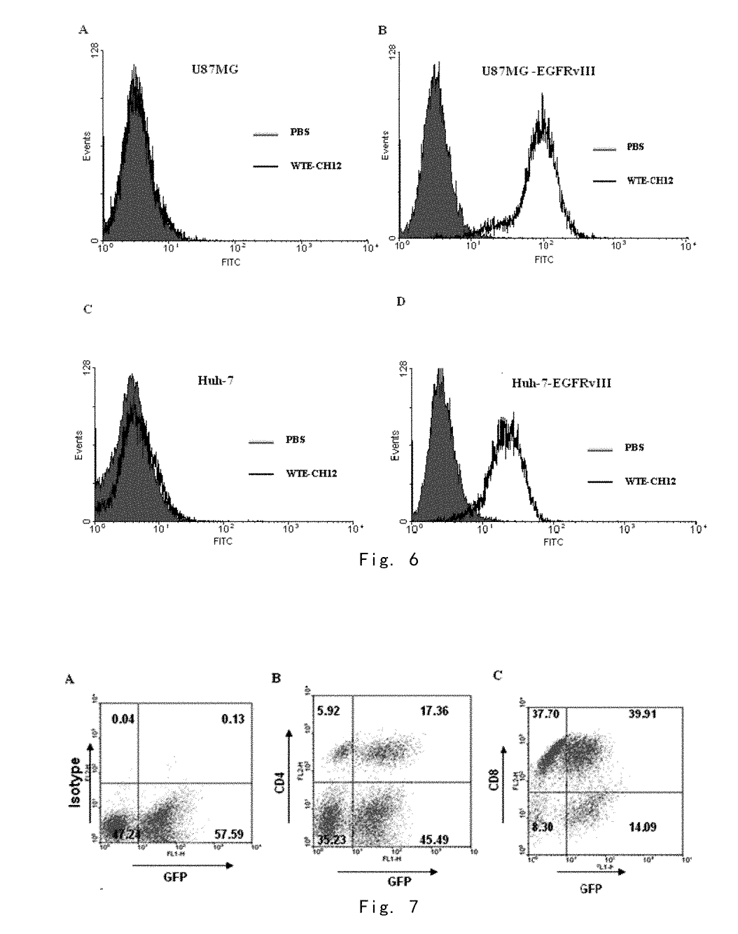

As shown in FIG. 6A, the antibody hardly binds to U87MG cells. As shown in FIG. 6B, the fluorescence peak of WTE-CH12 antibody displayed in black showed a significant difference compared with the blank control (PBS), indicating its ability to efficiently bind to U87MG-EGFRvIII cells. These results indicate that WTE-CH12 antibody can specifically bind to tumor cells overexpressing human U87MG-EGFRvIII.

As shown in FIG. 6C, the antibody hardly binds to Huh-7 cells. As shown in FIG. 6D, the fluorescence peak of WTE-CH12 antibody displayed in black showed a significant difference compared with the blank control (PBS, gray shade below the peak in the figure), indicating its ability to efficiently bind to Huh-7-EGFRvIII cells. These results indicate that WTE-CH12 antibody can specifically bind to tumor cells overexpressing human Huh-7-EGFRvIII.

### Example 4. Obtaining Sequence of Anti-WTE Polypeptide 2D8 Single Chain Antibody And Detection of Activities Thereof

1. Obtaining Nucleic Acid of Anti-WTE Polypeptide 2D8 Single Chain Antibody

The first strand of cDNA was synthesized by reverse transcription using RT-PCR Kit with the mRNA of hybridoma 2D8 cell strain against WTE (obtained from Shanghai Ruijin Biotechnology Co., Ltd.) as the template. VH and VL genes were amplified by using Heavy Primers and Light Primer Mix as the primers (primers were purchased from Shanghai Ruijin Biotechnology Co., Ltd.) and the first strand of cDNA as the template. Conditions for PCR were: pre-denaturation at 94° C. for 4 min; denaturation at 94° C. for 40 s; annealing at 55° C. for 40 s; extension at 68° C. for 40 s; 30 cycles; followed by extension at 68° C. for 7 min. Agarose gel electrophoresis detection of PCR products, and VH, VL fragments were recovered by gel recovery kit.

And then, VH and VL fragments were spliced by overlapping PCR to form scFv by using VH and VL fragments as the templates and Linker-Primer Mix as primers (primers were purchased from Shanghai Rui Jin Biotechnology Co., Ltd.). Conditions for PCR were: denaturation at 94° C. for 1 min; extension at 63° C. for 4 min; 7 cycles. After 7 cycles, Linker-Primer Mix, polymerase buffer and double distilled water were supplemented into 50 μl reaction system, and PCR was continued. Conditions for PCR were: pre-denaturation at 94° C. for 4 min; denaturation at 94° C. for 40 s; annealing at 58° C. for 40 s; extension at 68° C. for 1 min; 30 cycles; followed by extension at 68° C. for 7 min. Agarose gel electrophoresis detection of PCR products, and scFv fragments were recovered by gel recovery kit.

2. Expression of Anti-WTE Polypeptide 2D8 Single Chain Antibody and Detection of Activities Thereof

scFv fragments obtained in the above step and pCANTAB 5E vector (purchased from Pharmacia) were subject to double digestion by Sfi I and Not I, and digested fragments were recovered. The fragments were ligated at 16° C. overnight, and transformed into competent E. coli HB2151. Next day, 20 monoclones were picked from the transformation plate and cultured at 30° C. When OD600 reached 0.4˜0.6, a final concentration of 0.05 mmol/L of IPTG was added for inducing expression overnight (18 h). The supernatant was collected by centrifugation and the expression of soluble scFv in the culture supernatant was analyzed by ELISA. Specifically, 96-well plates were coated with antigen WTE-BSA (manufactured by Shanghai Ruijin Biotech Co., Ltd.) at 50 ng/well (1 ng/μl, 50 μl/well), incubated at 37° C. for 2 h, blocked with skim milk powder (Bright Dairy Co., Ltd.) in 5% PBS at 37° C. for 2 h, and washed for three times with 0.1 M phosphate buffer (PBS). The supernatant of the above culture for inducing expression was added into a 96-well plate, 50 μl per well, and incubate at 37° C. for 1 hour. After washing for 3 times with PBST (PBS+0.05% Tween 20), HRP-labeled anti-E tag antibody (purchased from Shanghai Ruijin Biotech Co., Ltd.) was diluted at 1:1000, 50 μl/well, and incubated at 37° C. for 1 h. After washing for 3 times with PBST, goat anti-mouse IgG-HRP diluted at 1:1000 (purchased from Santa Cruz) was added and incubated at 37° C. for 1 h. After washing for 5 times with PBST, ABTS color developing solution was added, 100 μL/well, and developed at 37° C. in darkness for 10 min. The absorbance value was measured by using Bio-Rad Model 680 microplate reader at a wavelength of 405 nm, and if the measured absorbance value was two times higher than that of the negative control, it was judged as positive.

Clone 2D8-3 with the highest OD value was sequenced, the sequence of single-chain antibody (scfv) of 2D8-3 was determined as SEQ ID NO: 35. The plasmid pCANTAB 5E 2D8-3 scfv was extracted as a template for constructing a lentiviral plasmid expressing the chimeric antigen receptor of the present invention.

### Example 5. Construction of Lentiviral Plasmid Expressing the Chimeric Antigen Receptor of the Present Invention

The chimeric antigen receptor protein encoded by the nucleic acid of the invention may be a chimeric antigen receptor protein comprising an extracellular binding domain, a transmembrane domain, and an intracellular signal domain in the following order:

eGFP-F2A-2D8scFv (anti-WTE)-CD8 hinge region-CD28a-CD28b-CD137-CD3ζ, wherein F2A is a ribosomal skipping sequence 2A (F2A) from foot and mouth disease (FMDV), for achieving co-expression of eGFP and CAR. CD28a represents its transmembrane region, and the second CD28b represents its intracellular signal region. Specific steps are listed as follows:

1. Obtaining Nucleic Acid Fragments

(1) Amplification of 2D8 Single Chain Antibody scFv (2D8 scFv (Anti-WTE)) Sequence

Upstream primer 5′-gccggccgaggtccagctg-3′ (SEQ ID NO: 17) and downstream primer 5′-cgtggtccgttttatttccaac-3′ (SEQ ID NO:18) were used in the amplification with the recombinant plasmid pCANTAB 5E 2D8-3 scfv in Example 4 as a template, and the size of amplified bands of interest was 723 bp. Conditions for PCR amplification were: pre-denaturation at 94° C. for 4 min; denaturation at 94° C. for 40 s; annealing at 58° C. for 40 s; extension at 68° C. for 40 s; 27 cycles; followed by extension at 68° C. for 10 min. PCR-amplified bands were confirmed by agarose gel electrophoresis to comply with the predicted fragment size.

(2) Amplification of eGFP Sequence

eGFP sequence was PCR-amplified by using upstream primer 5′-gcaggggaaagaatagtagaca-3′ (SEQ ID NO: 19) and downstream primer 5′-caaagtctgtttcacgctactagctagtcgagatctgagtccggacttgtacagctcgtc-3′ (SEQ ID NO: 20) with pWPT-eGFP (obtained from University of Geneva, Switzerland; Dr. Didier Trono) as a template, and the size of amplified bands of interest was 1297 bp. Conditions for PCR amplification were: pre-denaturation at 94° C. for 4 min; denaturation at 94° C. for 40 s; annealing at 58° C. for 40 s; extension at 68° C. for 90 s; 27 cycles; followed by extension at 68° C. for 10 min. PCR-amplified bands were confirmed by agarose gel electrophoresis to comply with the predicted fragment size.

(3) Amplification of Nucleic Acid Sequence of Other Parts of Chimeric Antigen Receptor

Other parts of the chimeric antigen receptor protein and the hinge region connecting these parts were amplified as follows: 1 ml Trizol (Invitrogen) was added into 1×10⁷ healthy human peripheral blood mononuclear cells (provided by Shanghai Blood Center) for the lysis of cells; afterwards, total RNA was extracted by phenol-chloroform method; and cDNAs were prepared through reverse transcription by using ImProm-II™ Reverse Transcription Kit (Promaga).

(a) Amplification of CD8a Hinge Region-CD8 Transmembrane Domain

CD8α hinge region-CD8 transmembrane domain was amplified by using upstream primer 5′-ttggaaataaaacggaccacgacgccagcg-3′ (SEQ ID NO: 21) and downstream primer 5′-ggtgataaccagtgacaggag-3′ (SEQ ID NO: 22) with the above prepared cDNA as a template. PCR amplification conditions were: pre-denaturation at 94° C. for 4 min; denaturation at 94° C. for 30 s; annealing at 58° C. for 30 s; extension at 68° C. for 30 s; 25 cycles; followed by a total extension at 68° C. for 10 min. The amplified product was confirmed by agarose gel electrophoresis to comply with the theoretical size, 198 bp.

(b) CD28 Transmembrane Region-CD28 Intracellular Signal Region Fragment

CD28 transmembrane region-CD28 intracellular signal region fragment was amplified by using upstream primer 5′-gacttcgcctgtgatttttgggtgctggtggtggttgg-3′ (SEQ ID NO: 23) and downstream primer 5′-ctttctgccccgtttggagcgataggct-3′ (SEQ ID NO: 24). PCR amplification conditions were identical with the above, and the amplified product was confirmed by agarose gel electrophoresis to comply with the theoretical size, 465 bp.

(c) CD137 Intracellular Signal Region

CD137 intracellular signal region was amplified by using upstream primer 5′-aaacggggcagaaagaaactc-3′ (SEQ ID NO: 25) and downstream primer 5′-cagttcacatcctccttc-3′ (SEQ ID NO: 26). PCR amplification conditions were identical with the above, and the amplified product was confirmed by agarose gel electrophoresis to comply with the theoretical size, 126 bp.

(d) CD3ζ Signal Region

CD3ζ zeta signal region was amplified by using upstream primer 5′-gaaggaggatgtgaactgagagtgaagttcagcaggagc-3′ (SEQ ID NO: 27) and downstream primer 5′-cgaggtcgacctagcgagggggcagggcctgcatg-3′ (SEQ ID NO: 28). PCR amplification conditions were identical with the above, and the amplified product was confirmed by agarose gel electrophoresis to comply with the theoretical size, 339 bp.

(e) Splicing of F2A-CD8α Signal Peptide Fragment Using Following Primers:

(SEQ ID NO: 29)
5′-actagctagtagcgtgaaacagactttgaattttgaccttctgaagt
tggc-3′;
(SEQ ID NO: 30)
5′-tggtaaggccatgggcccagggttggactcaacgtctcctgccaact
tcagaa-3′;
(SEQ NO: ID NO: 31)
5′-ccatggccttaccagtgaccgccttgctcctgccgctggccttgctg
ctccacgccgc-3′;
(SEQ ID NO: 32)
5′-gctggacctcggccggcctggcggcgtggagcag-3′.

Overlap PCR was employed, wherein SEQ ID NO: 29-SEQ ID NO: 32 were used as primers for synthesizing F2A-CD8α signal peptide fragment. In the first step, Overlap PCR amplification conditions were: pre-denaturation at 94° C. for 4 min; denaturation at 94° C. for 40 s; annealing at 58° C. for 40 s; extension at 68° C. for 40 s; 7 cycles; followed by a total extension at 68° C. for 5 min. In the second step, PCR was performed, wherein products from Overlap PCR in the first step were used as templates, and SEQ ID NO: 29 and SEQ ID NO: 32 were used as upstream and downstream primers respectively for amplifying F2A-CD8α signal peptide fragment. PCR amplification conditions were: pre-denaturation at 94° C. for 4 min; denaturation at 94° C. for 40 s; annealing at 58° C. for 40 s; extension at 68° C. for 30 s; 27 cycles; followed by a total extension at 68° C. for 5 min. The size of the amplified product was 142 bp, which was in agreement with the expected size.

2. Splicing of Nucleic Acid Fragment

(1) Splicing of CD137 Intracellular Signal Region-CD3ζ Fragment

Upstream primer 5′-gccccaccacgcgacttcgcagcctatcgctccaaacggggcagaaag-3′ (SEQ ID NO: 33) and downstream primer 5′-cgaggtcgacctagcgagggggcagggcctgcatg-3′ (SEQ ID NO: 34) were used in splicing CD137 intracellular signal region and CD3ζ signal region obtained through above mentioned amplification, that is, BBZ (abbreviated as CD137-CD3). Splicing and PCR amplification conditions were identical with the above, and the amplified product was confirmed by agarose gel electrophoresis to comply with the theoretical size, 512 bp.

(2) Splicing of CD8 Hinge Region-CD28 Transmembrane Region (Abbreviated as CD28a)-CD28 Intracellular Signal Region (Abbreviated as CD28b) Fragment

Upstream primer 5′-ttggaaataaaacggaccacgacgccagcg-3′ (SEQ ID NO: 21) and downstream 5′-ctttctgccccgtttggagcgataggct-3′ (SEQ ID NO: 24) primer were used in splicing CD8 hinge region obtained in (a) and CD28 transmembrane region-CD28 intracellular signal region obtained in (b) to obtain the target fragment: CD8 hinge region-CD28a-CD28b fragment. Splicing and PCR amplification conditions were identical with the above, and the amplified product was confirmed by agarose gel electrophoresis to comply with the theoretical size, 369 bp.

(3) Splicing of CD8 Hinge Region-CD28a-CD28b-CD137-CD3ζ Fragment

Upstream primer 5′-ttggaaataaaacggaccacgacgccagcg-3′ (SEQ ID NO: 21) and downstream primer 5′-cgaggtcgacctagcgagggggcagggcctgcatg-3′ (SEQ ID NO: 34) were used in splicing CD137-CD3ζ obtained in (1) and CD8 hinge region-CD28a-CD28b obtained in (2) by Overlap PCR to obtain the target fragment: CD8 hinge region-CD28a-CD28b-CD137-CD3. Splicing and PCR amplification conditions were identical with the above, and the amplified product was confirmed by agarose gel electrophoresis to comply with the theoretical size, 832 bp.

(4) Splicing of F2A-CD8α Signal Peptide and 2D8 scFv (Anti-WTE) Fragment:

Upstream primer 5′-actagctagtagcgtgaaacagactttgaattttgaccttctgaagttggc-3′ (SEQ ID NO: 29) and downstream primer 5′-cgtggtccgttttatttccaac-3′ (SEQ ID NO: 18) were used in splicing above obtained F2A-CD8α signal peptide and 2D8 scfv (anti-WTE) fragment obtain the target fragment: F2A-CD8α signal peptide-2D8 scfv. Splicing and PCR amplification conditions were identical with the above, and the amplified product was confirmed by agarose gel electrophoresis to comply with the theoretical size, 871 bp.

(5) Splicing of eGFP-F2A-CD8a-2D8 scFv (Anti-WTE)-CD8 Hinge Region-CD28a-CD28b-CD137-CD3ζ Fragment

Upstream primer 5′-gcaggggaaagaatagtagaca-3′ (SEQ ID NO: 19) and downstream primer 5′-tagcgtaaaaggagcaacatag-3′ (SEQ ID NO: 34) were used in splicing eGFP, F2A-CD8α signal peptide-2D8 scfv, CD8 hinge region-CD28a-CD28b-CD137-CD3ζ to obtain eGFP-F2A-CD8α-2D8scFv(WTE)-CD8 hinge region-CD28a-CD28b-CD137-CD3ζ Splicing conditions were: eGFP 65 ng+F2A-CD8α-2D8 scFv (anti-WTE) 50 ng+CD8 hinge region-CD28a-CD28b-CD137-CD3ζ 85 ng (molar ratio 1:1:1) pre-denaturation at 94° C. for 4 min; denaturation at 94° C. for 30 s; annealing at 60° C. for 30 s; extension at 68° C. for 30 s; 7 cycles; followed by a total extension at 68° C. for 10 min. DNA polymerase and upstream and downstream primers were supplemented, and afterwards PCR amplification was performed for 27 cycles; and amplification conditions were: pre-denaturation at 94° C. for 4 min; denaturation at 94° C. for 30 s; annealing at 60° C. for 30 s; extension at 68° C. for 120 s; 25 cycles; followed by a total extension at 68° C. for 10 min. The amplified product was confirmed by agarose gel electrophoresis to comply with the theoretical size, 2910 bp.

3. Construction of Plasmid Vector pWPT/eGFP-F2A-CD8α-2D8scFv (Anti-WTE)-CD28a-CD28b-CD137-CD3ζ

MluI and SalI cleavage sites were introduced upstream and downstream of the open reading frame of eGFP-F2A-CD8α-2D8 scFv (anti-WTE)-CD28a-CD28b-CD137-CD3ζ obtained in “2” by splicing. The obtained target gene was subject to double-digestion of MluI and SalI, and ligated into pWPT vector which was subject to the same double-digestion (see Huamao Wang., et al., Epidermal growth factor receptor vIII enhances tumorigenicity and resistance to 5-fluorouracil in human hepatocellular carcinoma. Cancer Letters 279 (2009) 30-38). The sequence of the recombinant plasmid was determined as correct for lentivirus packaging, and the pattern of the plasmid can be found in FIG. 4.

### Example 6. Preparation of Lentivirus for Infecting T Lymphocyte

1. Packaging of Lentivirus for Infecting T Lymphocyte

In this example, 293T cells were used in the packaging of lentivirus. In particular, 293T cells (ATCC: CRL-11268) which were cultured to 10th-20th generation were inoculated into a 10 cm Petri dish at a density of 5×10⁶, and cultured overnight at 37° C., 5% CO₂ for transfection. The medium was DMEM (PAA) containing 10% fetal bovine serum (PAA). The next day, the medium was replaced with serum-free DMEM at about 2 hours before transfection. The procedure for transfection is listed as follows: 20 μg of target gene plasmid pWPT/eGFP-scFv (anti-WTE)-CD28a-CD28b-CD137-CD3ζ was homogeneously mixed with 15 μg of packaging plasmid PAX2 and 6 μg of envelope plasmid pMD2.G (see Huamao Wang., et al., Epidermal growth factor receptor vIII enhances tumorigenicity and resistance to 5-fluorouracil in human hepatocellular carcinoma. Cancer Letters 279 (2009) 30-38) into 500 μL of MillQ water. 62 μL of 2.5 M CaCl₂ (Sigma) was added dropwise and mixed at 1200 rpm/min vortex. Finally, 500 μL of 2×HeBS (280 mM NaCl, 10 mM KCl, 1.5 mM Na₂HPO₄.2H₂O, 12 mM glucose, 50 mM Hepes (Sigma), pH 7.05, 0.22 μM, filtered for sterilization) was added dropwise. The obtained solution was immediately added into the Petri dish dropwise, gently stirred, incubated at 37° C., 5% CO₂ for 4-6 h, and replaced with DMEM containing 10% fetal bovine serum. Transfection efficiency was observed on the next day (i.e., the proportion of cells with green fluorescence), and about 80% of positive transfection efficiency was deemed as successful transfection. After 48 h or 72 h of transfection, virus was collected by filtration using a 0.45 μm filter (Millipore) and stored at −80° C.

2. Purification of Lentivirus for Infecting T Lymphocyte and Determination of Titer Thereof

(1) Purification of Lentivirus for Infecting T Lymphocyte

The virus supernatant collected in the above procedure was centrifuged at 28000 rpm for 2 hours at 4° C. using a Beckman Optima L-100XP ultracentrifuge. The obtained supernatant was discarded, and the resulting pellet was resuspended into 1/10 to 1/30 volume of initial solution of Quantum 007 medium (PAA), and frozen at −80° C. at 100 μl/tube.

(2) Determination of Titer of Lentivirus for Infecting T Lymphocyte

The method for determination of lentivirus titer is listed as follows: 293T cells were inoculated in a 96-well culture plate at 1×10⁵/mL, 50 μl/well. The medium is DMEM containing 10% fetal calf serum. Virus concentrate was added to each well, 5 μl/well and the final volume was supplemented to 50 μl. Each sample was 3-fold diluted (6 gradients, and duplicate wells). Gradient dilution of virus was homogeneously mixed cells, and incubated at 37° C., 5% CO₂. 48 h after infection, eGFP was detected by flow cytometer. The number of cells, when the positive rate is 5 to 20%, is appropriate, and the titer (U/mL) was calculated as positive rate×fold of dilution×100×10⁴.

### Example 7. Sorting CD4⁺ or CD8⁺ T Lymphocyte and Infection of Lentivirus

1. Sorting CD4⁺ or CD8⁺ T Lymphocyte

Peripheral blood mononuclear cells from healthy people (provided by Shanghai Blood Center) were sorted by using CD4⁺ or CD8⁺ T lymphocyte sorting beads (Stem Cell Technologies) to obtain CD4⁺ or CD8⁺ T lymphocyte, specific procedure of which can be found in the instruction.

Upon sorting, CD4⁺ and CD8⁺ T lymphocytes were mixed at 1:1, and added into Quantum 007 lymphocyte culture medium (PAA) at a density of 1×10⁶/mL. Magnetic beads (Invitrogen) simultaneously coated with anti-CD3ζ and anti-CD28 antibodies at cell: magnetic bead of 1:1 and recombinant human IL-2 at a final concentration of 100 U/mL (Shanghai Huaxin Biotechnology Co., Ltd.) were added, and cultured at 37° C., 5% CO₂ for 24 h.

2. Infection of CD4⁺ or CD8⁺ T Lymphocytes by Lentivirus and Detection of Positive Rate

Cells obtained by sorting were cultured for 24 h, and CD4⁺ or CD8⁺ lymphocytes were infected by recombinant lentivirus at MOI=5. The infected T lymphocytes were subcultured at a density of 5×10⁵/mL with a culture density being not higher than 2×10⁶/mL, and recombinant human IL-2 at a final concentration of 100 U/mL was supplemented. Infected T-lymphocytes were tested for positive rate of target gene by flow cytometer at one day before the next experiment. The detected eGFP-positive cells are positive cells expressing chimeric antigen receptor, since eGFP and CAR were co-expressed. Positive rate of transfection is 57.9%, as shown in FIG. 7A.

The proportion of CD4⁺ eGFP⁺ and CD8⁺ eGFP⁺ cells in mix-infected T lymphocytes was determined by a flow cytometer. In particular, the infected T lymphocytes were collected by centrifugation at 200 g×5 min, resuspended in 1% phosphate buffer (NBS PBS) containing calf bovine serum at a cell density of 5×10⁶/mL, and added into flow tubes at an amount of 100 μI/tube. Blank control PBS, 1:50 dilution of anti-CD4 mouse monoclonal antibody and anti-CD8 mouse monoclonal antibody (from Santa Cruz) were added into 3 tubes (100 μI/tube), respectively, and incubated in an ice bath. After 45 mins, 2 ml of 1% NBS PBS was added into each tube, and centrifuged at 200 g×5 min for two times. The supernatant was discarded, and 1:50 dilution of goat-anti-mouse-PE mouse monoclonal antibody (from Santa Cruz) were added (100 μI/tube), and incubated in an ice bath. After 45 mins, 2 ml of 1% NBS PBS was added into each tube, and centrifuged at 200 g×5 min for two times. The supernatant was discarded, and the pellet was resuspended into 300 μl of 1% NBS PBS and detected by a flow cytometer. Data were analyzed using WinMDI 2.9 (a flow cytometric data analysis software). Results can be found in FIGS. 7B and 7C, wherein the proportion of CD4⁺ eGFP⁺ and CD8⁺ eGFP⁺ cells are 17.6% and 39.9%, respectively.

### Example 8. In Vitro Toxicity Experiment of T Lymphocytes Expressing Chimeric Antigen Receptor Mediated by WTE-CH12

In the in vitro toxicity experiment, target cells are U87MG, U87MG-EGFRvIII, Huh-7, Huh-7-EGFRvIII, respectively; and effector cells were cells cultured in vitro for 12 days and detected as chimeric antigen receptor-positive cells by FACS, i.e., chimeric antigen receptor-positive T lymphocytes (CAR⁺CD4⁺ and CAR⁺CD8⁺ mixed cells). The action ratio of U87MG, U87MG-EGFRvIII effector cells to target cells was 10:1, that of Huh-7, Huh-7-EGFRvIII effector cells to target cells was 3:1, and the amount of target cells was 10000/well. In experimental groups, the maximal concentration of WTE-CH12 antibody was 10⁴ ng/ml, and ten-fold diluted in 4 gradients respectively. Quintuplicate wells were set for each concentration in the experimental group and the control group, and the average of quintuplicate wells was taken. The detection was performed at the 18^{th} hour. Wherein the experiment group and control groups are listed as follows:

Experimental group: target cells+chimeric antigen receptor-positive T lymphocytes+WTE-CH12 antibody

Control group 1: target cells with maximum release of LDH,

Control group 2: target cells spontaneously releasing LDH,

Control group 3: effector cells+target cells.

Specific detection method were carried out according to CytoTox 96 Non-radioactive Cytotoxicity Assay Kit (Promega). The method is based on the colorimetric method, which can replace 51Cr release method. CytoTox 96® assay quantitatively measures lactate dehydrogenase (LDH). LDH is a stable cytoplasmic enzyme that is released during cell lysis and is released in the same manner as 51Cr in radioactivity analysis. Released LDH is present in the culture supernatant and can be detected by a 30-minute coupled enzymatic reaction, in which LDH converts a tetrazolium salt (INT) into a red formazan, and the amount of the resulting red product is proportional to the number of lysed cells.

Cytotoxicity was calculated as:

Cytotoxicity %=[(Experimental group−Control group 3)/(Control group 1−Control group 2)]×100%

The experimental results showed that, in the presence of WTE-CH12 antibody, 2D8 scFv (anti-WTE)-CD28a-CD28b-CD137-CD3CAR⁺ lymphocytes (CD4⁺ and CD8⁺ mixed lymphocytes) exhibited significant cytotoxicity to tumor cells U87MG-EGFRvIII and Huh-7-EGFRvIII. The produced cytotoxicity effect significantly depends on the concentration gradient of the antibody, and the cell killing effect is up to 98.3% and 93.0% when the concentration of antibody is 10⁴ ng/ml, respectively. However, the cytotoxicity to U87MG cells is not significant under the same condition (3.28%), there are certain killing effects to Huh-7 cells when the concentration is 10²-10⁴ ng/ml, while there are remarkable killing effects to Huh-7-EGFRvIII cells at the same concentration. Particular results can be found in FIG. 8.

### Example 9. In Vitro Competitive Inhibition Experiment of WTE Polypeptide on Toxic Effects of T Lymphocytes Expressing Chimeric Antigen Receptor on Tumor Cells Mediated by WTE-CH12

In the in vitro toxicity competitive inhibition experiment, target cells are U87MG-EGFRvIII, and effector cells were T lymphocytes cultured in vitro for 12 days and detected as chimeric antigen receptor-positive cells by FACS, i.e., chimeric antigen receptor-positive T lymphocytes (CAR⁺CD4⁺ and CAR⁺CD8⁺ mixed cells). The action ratio of U87MG-EGFRvIII effector cells to target cells was 10:1, and the amount of target cells was 10000/well. In experimental groups, the concentration of WTE-CH12 antibody was 10⁴ ng/ml, and no WTE-CH12 antibody was added in Control group. Quintuplicate wells were set for each concentration in the experimental group and control group, and the average of quintuplicate wells was taken. The detection was performed at the 18^{th} hour. Wherein the experiment group and control groups are listed as follows:

Experimental group 1: Target cells+chimeric antigen receptor-positive T lymphocytes+WTE-CH12 antibody

Experimental group 2: Target cells+chimeric antigen receptor-positive T lymphocytes+WTE-CH12 antibody+WTE polypeptide (2-fold molar concentration of WTE-CH12 antibody)

Experimental group 3: Target cells+chimeric antigen receptor-positive T lymphocytes+WTE-CH12 antibody+WTE polypeptide (20-fold molar concentration of WTE-CH12 antibody) Control group 1: target cells with maximum release of LDH,

Control group 2: target cells spontaneously releasing LDH,

Control group 3: effector cells+target cells.

Particular test method and calculation formula can be found in Example 8.

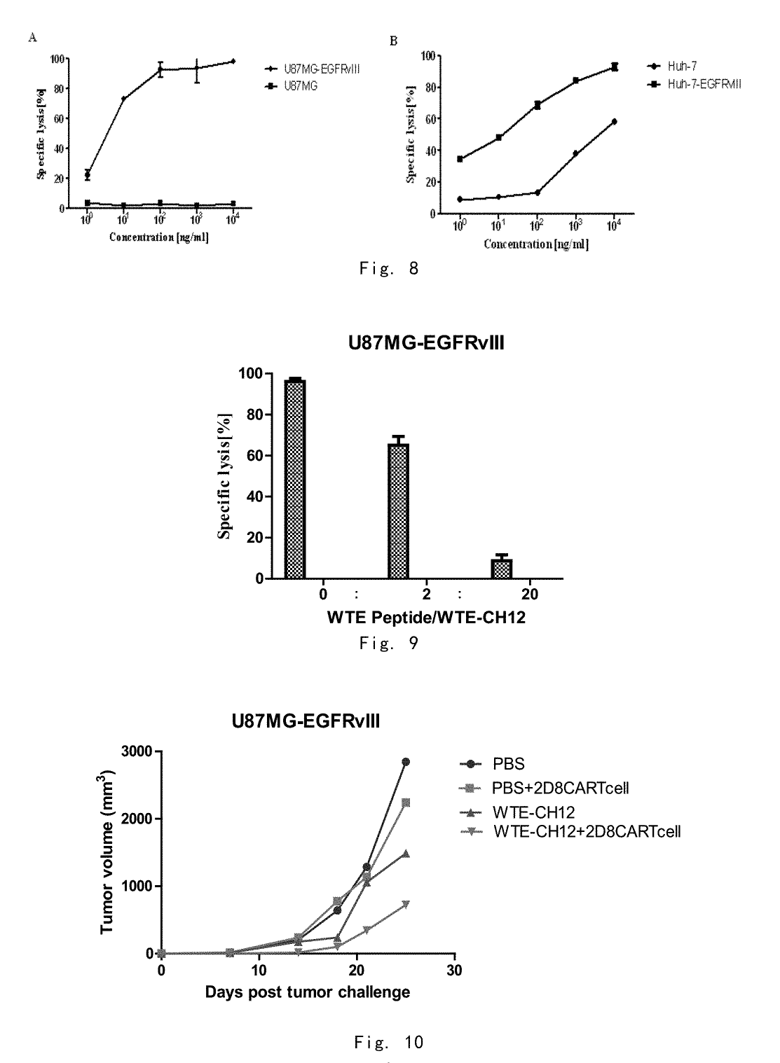

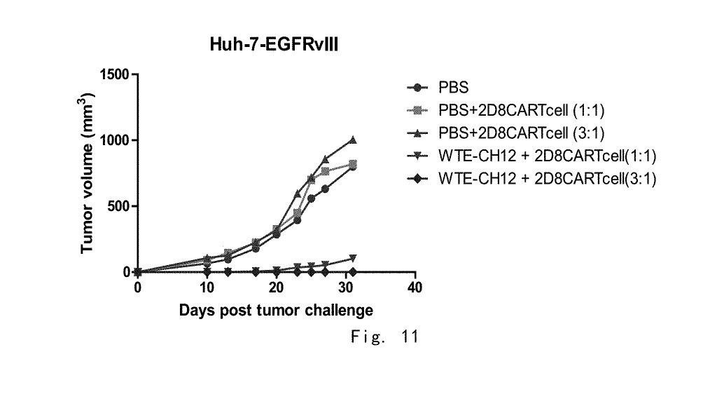

The experimental results showed that free WTE polypeptides showed a certain inhibitory effect on the WTE-CH12 antibody-mediated toxic effects of CAR⁺ T cells, when the molar concentration of the free WTE polypeptide was 2 times of that of WTE-CH12 antibody. And the inhibitory effect was reduced by 31.1%, compared with Experimental group 1, and when the molar concentration of the free WTE polypeptide was 20 times of that of WTE-CH12 antibody, the inhibitory effect on the WTE-CH12 antibody-mediated toxic effects of CAR⁺ T cells is significant, which was reduced by 87.83, compared with Experimental group 1. Particular results can be found in FIG. 9.

### Example 10. In Vivo Antitumor Activity of T Lymphocytes Expressing Chimeric Antigen Receptor Mediated by WTE-CH12 in Tumor-Bearing Mice

1. In Vivo Anti-Tumor Activity of CAR T Cells Mediated by WTE-CH12 in Tumor-Bearing Mice (U87MG-EGFRvIII)

6-10 week-old immunodeficient NOD/SCID mice (provided by Shanghai Slack Laboratory Animal Co., Ltd.) were used to construct a xenograft model of human EGFR-related tumor, genetic characters of which are lacking of T cells, B cells, NK cells as well as macrophage function. In the experiment, the number of inoculated cells was 5×10⁵/animal for U87MG-EGRFRvIII, 5×10⁶/animal for CAR⁺ T cell, and 50 μg/animal for WTE-CH12 antibody. The experiment groups are listed as follows:

1: U87MGEGFRVIII+PBS

2: U87MGEGFRVIII+PBS+CAR T cell

3: U87MGEGFRVIII+WTE-CH12 (50 μg)

4: U87MGEGFRVIII+CAR T cell+WTE-CH12 (50 μg).

In particular, 6 to 8 week-old mice were divided into groups (6 mice per group) as mentioned above, and 100 mg/kg of cyclophosphamide (working solution 20 mg/ml, working dose 5 μl/g of mice) was intraperitoneally given at the day before inoculation of cells. Next day, a suspension of U87MG-EGFRvIII cells (2.5×10⁶/ml, 200 μl) was subcutaneously inoculated in the right side of mice in groups 1 and 3, and a mixed suspension of U87MG-EGFRvIII and CART cells was subcutaneously inoculated in the right side of mice in groups 2, 4 and 5, wherein the suspension was obtained by mixing 100 μl of U87MG-EGFRvIII (concentration of which was 5×10⁶/ml) and 100 μl of CAR⁺ T cells (concentration of which was 5×10⁷/ml) at a volume ratio of 1:1. One hour after cell inoculation, mice in groups 1 and 2 were injected with PBS (100 μl) via tail vein, and mice in group 3 and 4 were injected with 50 μg of WTE-CH12 antibody (0.5 mg/ml, 100 μl) respectively.

On a designated day, the size of the tumor was measured by a vernier caliper, and the tumor volume was calculated according to the following formula:

Tumor volume=(length×width×width)/2

The reduction of tumor volume in the mouse model was set as the basis for the inhibitory effects of WTE-CH12-mediated CAR⁺ T cells on tumor. The tumor inhibition rate was calculated as follows:

Tumor inhibition rate=1−(tumor volume in treatment group−tumor volume in control group)×100%

The results are shown in FIG. 10, wherein, in the U87MG-EGFRvIII tumor-bearing mouse model, no significant intervention on the growth of U87MG-EGFRvIII tumor was observed for the mice in control group 2 (merely injected with U87MG-EGFRvIII tumor cells and CAR T effector cells), compared with control group 1 (merely injected with tumor cells) at 25 days after cell inoculation, and the inhibition rate was 21.2%. Compared with control group 2, there was a certain anti-tumor effect in control group 3 (merely injected with tumor cells and WTE-CH12 antibody) and the inhibition rate was 33.5%. However, such effect is significantly different from that of experimental group 4 (injected with tumor cells, effector cells and WTE antibody), the inhibition rate of which is 67.6%. The results showed that, CART cells expressing anti-WTE polypeptide single-chain antibody exhibits strong ability to inhibit the growth of U87MG-EGFRvIII mediated by WTE-CH12 antibody.

2. In Vivo Anti-Tumor Activity of CAR T Cells Mediated by WTE-CH12 in Tumor-Bearing Mice (Huh-7-EGFRVIII)

The mice used in the experiment were the same as described above. In the experiment, the number of inoculated Huh-7-EGRFRvIII cells was 3×10⁶/animal, the number of inoculated CAR⁺ T cells was 3×10⁶/animal (the ratio of effector cell to target cell was 1:1) and 9×10⁶/animal (the ratio of effector cell to target cell was 3:1) respectively, and WTE-CH12 antibody was injected at 50 μg/animal. The experiment groups are listed as follows:

1: Huh-7-EGFRvIII+PBS;

2: Huh-7-EGFRvIII+CAR T+PBS (the ratio of effector cell to target cell was 1:1);

3: Huh-7-EGFRvIII+CAR T+WTE-CH12 (50 μg) (the ratio of effector cell to target cell was 1:1);

4: Huh-7-EGFRvIII+CAR T+PBS (the ratio of effector cell to target cell was 3:1);

5: Huh-7-EGFRvIII+CAR T+WTE-CH12 (50 μg) (the ratio of effector cell to target cell was 3:1).

In particular, 6 to 8 week-old mice were divided into groups (5 mice per group) as mentioned above, and 100 mg/kg of cyclophosphamide (working solution 20 mg/ml, working dose 5 μl/g of mice) was intraperitoneally given at the day before inoculation of cells. Next day, a suspension of Huh-7-EGFRvIII cells (1.5×10⁷/ml, 200 μl) was subcutaneously inoculated in the right side of mice in group 1, a mixed suspension of Huh-7-EGFRvIII and CART cells was subcutaneously inoculated in the right side of mice in groups 2, and 3, wherein the suspension was obtained by mixing 100 μl of Huh-7-EGFRvIII (concentration of which was 1.5×10⁷/ml) and 100 μl of CAR⁺ T cells (concentration of which was 1.5×10⁷/ml) at a volume ratio of 1:1, and a mixed suspension of Huh-7-EGFRvIII and CART cells was subcutaneously inoculated in the right side of mice in groups 4, and 5, wherein the suspension was obtained by mixing 100 μl of Huh-7-EGFRvIII (concentration of which was 1.5×10⁷/ml) and 100 μl of CAR⁺ T cells (concentration of which was 4.5×10⁷/ml) at a volume ratio of 1:1. One hour after cell inoculation, mice in groups 1, 2 and 4 were injected with PBS (100 μl) via tail vein, and mice in group 3 and 5 were injected with 50 μg of WTE-CH12 antibody (0.5 mg/ml, 100 μl) respectively. The tumor volume was measured and the tumor inhibition rate was calculated as described in Example 10-1.

The results are shown in FIG. 11, wherein, in the Huh-7-EGFRvIII tumor-bearing mouse model, compared with 1:1 control group 2 (merely injected with tumor cells and effector cells), there was a significant intervention on the growth of Huh-7-EGFRvIII tumor in 1:1 experiment group 3 (injected with tumor cells, effector cells and WTE-CH12 antibody), and the inhibition rate was 87.5%. Compared with 3:1 control group 4 (merely injected with tumor cells and effector cells), there was a significant inhibition on the growth of Huh-7-EGFRvIII tumor in 3:1 experiment group 5 (injected with tumor cells, effector cells and WTE-CH12 antibody), and the inhibition rate was 100%.

All documents mentioned in the present invention are hereby incorporated by reference as if each individual document was individually incorporated by reference. It is also to be understood that various changes or modifications can be made to the invention by those skilled in the art upon reading the contents of the present invention, and such equivalents fall within the scope of the claims appended hereto.

1. A system for inhibiting pathological target cells, comprising:
(1) a fusion protein, comprising a polypeptide tag and a binding molecule which specifically recognizes pathogenic target cells; and
(2) a chimeric antigen receptor immune effector cell, expressing binding molecules which specifically recognize the polypeptide tag.

2. The system of claim 1, wherein the polypeptide tag is an unrelated antigen with low immunogenicity, which is of low or non-expression in non-tumor tissue; preferably, the polypeptide tag is a WTE tag.

3. The system of claim 1, wherein the pathological target cell is a tumor cell, and the binding molecule that specifically recognizes the pathological target cell binds to tumor-associated antigen on the tumor cell; preferably, the tumor-associated antigen is selected from:
EGFR, EGFRvIII, de4 EGFR, EpCAM, CD19, CD20, CD33, HER2, EphA2, IL13R, GD2, LMP1, Claudin 18.A2, PLAC1, NY-ESO-1, MAGE4, MUC1, MUC16, LeY, CEA, GPC3, Mesothelin, CAIX, CD123, IL13R, EphA2.

4. The system of claim 1, wherein the immune effector cell includes: T lymphocyte, NK cell.

5. The system of claim 1, wherein the pathological target cells are tumor cells that express EGFRvIII; and
the binding molecule that specifically recognizes pathogenic target cells is an antibody that specifically binds EGFRvIII.

6. The system of claim 1, wherein the chimeric antigen receptor immune effector cell recombinantly expresses one or more selected from CD28, CD137, CD3ζ, CD27, CD8, CD19, CD134, CD20, FcRγ.

7. Use of system of claim 1, for preparing a medical cartridge for inhibiting pathological target cells.

8. A kit for preparing the medical cartridge of claim 7, wherein the kit includes:
(a) expression construct a, comprising an expression cassette of a fusion protein, wherein the fusion protein comprises a polypeptide tag and a binding molecule that specifically recognizes pathological target cells;
(b) expression construct b, comprising an expression cassette which expresses a binding molecule that specifically recognizes the polypeptide tag; and
(c) immune effector cells.

9. An isolated polypeptide, wherein the amino acid sequence of the polypeptide is encoded by the nucleotide sequence of SEQ ID NO: 38.

10. A single chain antibody that specifically binds to the polypeptide of claim 9, wherein the single chain antibody is encoded by the nucleotide sequence of SEQ ID NO: 35.


SEQUENCE LISTING (40 sequences)

SEQ ID NO: 1 - DNA, 23 bp, Artificial Sequence
  synthetic primer
gatgtgcagcttcaggagtcggg

SEQ ID NO: 2 - DNA, 22 bp, Artificial Sequence
  synthetic primer
acaataatatgtggctgtgtcc

SEQ ID NO: 3 - DNA, 56 bp, Artificial Sequence
  synthetic primer
cctagctagccaccatgagagtgctgattcttttgtggctgttcacagcctttcct

SEQ ID NO: 4 - DNA, 46 bp, Artificial Sequence
  synthetic primer
agctgtggagccagacaggaaaccaggaaaggctgtgaacagccac

SEQ ID NO: 5 - DNA, 51 bp, Artificial Sequence
  synthetic primer
ggtttcctgtctggctccacagctgaaaatgcagaatacctaagggtcgcg

SEQ ID NO: 6 - DNA, 54 bp, Artificial Sequence
  synthetic primer
tgctccaataaattcactgctttgtggcgcgacccttaggtattctgcattttc

SEQ ID NO: 7 - DNA, 51 bp, Artificial Sequence
  synthetic primer
ccacaaagcagtgaatttattggagcagcatcaaccaaaggtcctgatgtg

SEQ ID NO: 8 - DNA, 36 bp, Artificial Sequence
  synthetic primer
ctcctgaagctgcacatcaggacctttggttgatgc

SEQ ID NO: 9 - DNA, 23 bp, Artificial Sequence
  synthetic primer
gacatcctgatgacccaatctcc

SEQ ID NO: 10 - DNA, 21 bp, Artificial Sequence
  synthetic primer
gaagacagatggtgcagccac

SEQ ID NO: 11 - DNA, 56 bp, Artificial Sequence
  synthetic primer
gatcgatatccaccatggacatgatggtccttgctcagtttcttgcattcttgttg

SEQ ID NO: 12 - DNA, 49 bp, Artificial Sequence
  synthetic primer
aaaccaaagcaacaagaatgcaagaaactgagcaaggaccatcatgtcc

SEQ ID NO: 13 - DNA, 52 bp, Artificial Sequence
  synthetic primer
ctttggtttccaggtgcaagatgtggctccacagctgaaaatgcagaatacc

SEQ ID NO: 14 - DNA, 57 bp, Artificial Sequence
  synthetic primer
tggcgcgacccttaggtattctgcattttcagctgtggagccacatcttgcacctgg

SEQ ID NO: 15 - DNA, 56 bp, Artificial Sequence
  synthetic primer
taagggtcgcgccacaaagcagtgaatttattggagcaacggtggctgcaccagac

SEQ ID NO: 16 - DNA, 57 bp, Artificial Sequence
  synthetic primer
ttgggtcatcaggatgtctggtgcagccaccgttgctccaataaattcactgctttg

SEQ ID NO: 17 - DNA, 19 bp, Artificial Sequence
  synthetic primer
gccggccgaggtccagctg

SEQ ID NO: 18 - DNA, 22 bp, Artificial Sequence
  synthetic primer
cgtggtccgttttatttccaac

SEQ ID NO: 19 - DNA, 22 bp, Artificial Sequence
  synthetic primer
gcaggggaaagaatagtagaca

SEQ ID NO: 20 - DNA, 60 bp, Artificial Sequence
  synthetic primer
caaagtctgtttcacgctactagctagtcgagatctgagtccggacttgtacagctcgtc

SEQ ID NO: 21 - DNA, 30 bp, Artificial Sequence
  synthetic primer
ttggaaataaaacggaccacgacgccagcg

SEQ ID NO: 22 - DNA, 21 bp, Artificial Sequence
  synthetic primer
ggtgataaccagtgacaggag

SEQ ID NO: 23 - DNA, 38 bp, Artificial Sequence
  synthetic primer
gacttcgcctgtgatttttgggtgctggtggtggttgg

SEQ ID NO: 24 - DNA, 28 bp, Artificial Sequence
  synthetic primer
ctttctgccccgtttggagcgataggct

SEQ ID NO: 25 - DNA, 21 bp, Artificial Sequence
  synthetic primer
aaacggggcagaaagaaactc

SEQ ID NO: 26 - DNA, 18 bp, Artificial Sequence
  synthetic primer
cagttcacatcctccttc

SEQ ID NO: 27 - DNA, 39 bp, Artificial Sequence
  synthetic primer
gaaggaggatgtgaactgagagtgaagttcagcaggagc

SEQ ID NO: 28 - DNA, 35 bp, Artificial Sequence
  synthetic primer
cgaggtcgacctagcgagggggcagggcctgcatg

SEQ ID NO: 29 - DNA, 51 bp, Artificial Sequence
  synthetic primer
actagctagtagcgtgaaacagactttgaattttgaccttctgaagttggc

SEQ ID NO: 30 - DNA, 53 bp, Artificial Sequence
  synthetic primer
tggtaaggccatgggcccagggttggactcaacgtctcctgccaacttcagaa

SEQ ID NO: 31 - DNA, 58 bp, Artificial Sequence
  synthetic primer
ccatggccttaccagtgaccgccttgctcctgccgctggccttgctgctccacgccgc

SEQ ID NO: 32 - DNA, 34 bp, Artificial Sequence
  synthetic primer
gctggacctcggccggcctggcggcgtggagcag

SEQ ID NO: 33 - DNA, 48 bp, Artificial Sequence
  synthetic primer
gccccaccacgcgacttcgcagcctatcgctccaaacggggcagaaag

SEQ ID NO: 34 - DNA, 35 bp, Artificial Sequence; identical to SEQ ID NO: 28
  synthetic primer

SEQ ID NO: 35 - DNA, 726 bp, Artificial Sequence
  synthetic single-chain antibody (scfv) polynucleotide
atggccgaggtccagctgcagcaatctggacctgagctggtgaagcctggggcttcagtgaggataccctgcaaggcttctggatacacattcactgactacaacatgggctgggtgaagcagagccatggaaagagccttgagtggattgcagatattaatcctaacaatggtcttactatctacaaccagaagttcaagggcaaggccacattgactgtagacaagtcctccaccacagcctacatggagctccgcagcctgacatctgaggacactgcagtctattactgttcaactgttacttactggggccaagggactctggtcactgtctctgcaggtggaggcggttcaggcggaggtggttctggcggtggcggatcggacattcagatgacccagactccactctccctgcctgtcagtcttggagatcaagcctccatctcttgcagatctagtcagagccttgtacacagtaatggaaacacctttttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaatgtacacatgttcctttcacgttcggctcggggacaaagttggaaataaaacgg

SEQ ID NO: 36 - DNA, 1395 bp, Artificial Sequence
  synthetic antibody pH CH12 polynucleotide
atgagagtgctgattcttttgtggctgttcacagcctttcctggtttcctgtctgatgtgcagcttcaggagtcgggacctggcctggtgaagccttctcagtctctgtccctcacctgcactgtcactgcctactcagtcaccagtgattatgcctggaactggatccggcagtttccaggaaacaaactggagtggatgggctacataagctacagtggtaccactagatacaacccatctctcaaaagtcgaatctctatcactcgagacacatccaagaaccagttcttcctgcagttgaattctatgactgctgaggacacagccacatattattgttcaagacagggacgggggtttccttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - DNA, 711 bp, Artificial Sequence
  synthetic antibody pK CH12 polynucleotide
  misc_feature: Polynucleotide
atggacatgatggtccttgctcagtttcttgcattcttgttgctttggtttccaggtgcaagatgtgacatcctgatgacccaatctccatcctccatgtctgtatctctgggagacacagtcagcatcacttgccatgcaagtcaggacattaacagtaatatagggtggttgcaacagaaaccagggaaatcatttaagggcctgatctatcatggaaccaacttggaagatggagttccatcaaggttcagtggcagtggatctggagcagattattctctcaccatcagcagcctggaatctgaagattttgcagactattactgtgtgcagtatgctcagtttccgtggacgttcggtggaggcaccaaactggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 38 - DNA, 66 bp, Artificial Sequence
  synthetic WTE fragment polynucleotide
ggctccacagctgaaaatgcagaatacctaagggtcgcgccacaaagcagtgaatttattggagca

SEQ ID NO: 39 - DNA, 54 bp, Artificial Sequence
  synthetic heavy chain signal polynucleotide
atgagagtgctgattcttttgtggctgttcacagcctttcctggtttcctgtct

SEQ ID NO: 40 - DNA, 66 bp, Artificial Sequence
  synthetic light chain signal polynucleotide
atggacatgatggtccttgctcagtttcttgcattcttgttgctttggtttccaggtgcaagatgt